(19) United States
(12) Patent Application Publication
(10) Pub. No.: US 2026/0167878 A1
(43) Pub. Date: Jun. 18, 2026

(54) PROCESS FOR PRODUCING WAX-BASED CHEMICALS AND ALPHA-OLEFINS FROM WASTE PLASTICS BASED ON SELF-HEATING MELT DECHLORINATION

(71) Applicant: China University of Petroleum (East China), Qingdao (CN)

(72) Inventors: Yingyun QIAO, Qingdao (CN); Yuanyu TIAN, Qingdao (CN); Peijie ZONG, Qingdao (CN); Jingxian WANG, Qingdao (CN); Yiliang TIAN, Qingdao (CN); Wenshuo MA, Qingdao (CN); Wenlong XU, Qingdao (CN); Hongbo CHEN, Qingdao (CN); Jie LI, Qingdao (CN); Fuqiang HAN, Qingdao (CN)

(21) Appl. No.: 19/421,858

(22) Filed: Dec. 16, 2025

(30) Foreign Application Priority Data
Dec. 17, 2024 (CN) 202411865261.X

(51) Int. Cl. C10G 55/06

(52) U.S. Cl. CPC C10G 55/06; C10G 2300/1003; C10G 2400/20; C10G 2400/22

(57) ABSTRACT

A process for producing wax-based chemicals and α-olefins from waste plastics based on self-heating melt dechlorination, wherein the clean fragments of waste plastics are added into a water-cooling feed pipe, then fall into a hot melting dechlorination reactor; a part of molten plastic liquid extracted from the bottom of said hot melting dechlorination reactor is returned to the upper part of said hot melting dechlorination reactor, the other part of the molten plastic liquid is used as a raw material and fed to an upper middle part of a downer bed catalytic pyrolysis reactor; a gas-solid separation is performed at the bottom of said downer bed catalytic pyrolysis reactor; the separated pyrolysis oil gas is separated into α-olefin fraction, wax oil, high temperature oil slurry, and pyrolysis dry gas, the high temperature oil slurry is directly recycled to the upper part of said hot melting dechlorination reactor.

### CROSS-REFERENCE TO RELATED APPLICATIONS

The application claims priority to Chinese Application No. 202411865261.X, filed on Dec. 17, 2024 and entitled “PROCESS FOR PRODUCING WAX-BASED CHEMICALS AND α-OLEFINS FROM WASTE PLASTICS BASED ON SELF-HEATING MELT DECHLORINATION”, the content of which is specifically and entirely incorporated herein by reference.

### FIELD

The present disclosure provides a process for producing wax-based chemicals and α-olefins from waste plastics based on self-heating melt dechlorination, which belongs to the technical field of environmental engineering.

### BACKGROUND

Although the widespread use of plastics has provided great convenience for daily life of the people, it has also brought forth a lot of white pollution. The use cycle of plastics is very short, a large number of plastic products, especially packaging materials, are discarded about 6-12 months after putting into use, 40% of plastics are discarded after 1-2 years, the total annual output of plastic waste in the world has reached 50 million tons. In the past few decades, waste plastics have been regarded as a part of municipal solid wastes (MSW). According to surveys, waste plastics account for 4%-10% (wt) or 10%-20% (v %) of MSW in the industrially developed countries, mainly derived from packing waste, automobile garbage and processing waste. The percentages of various types of waste plastics are as follows:27% of low-density polyethylene (LDPE), 21% of high-density polyethylene (HDPE), 18% of polypropylene (PP), 16% of polystyrene (PS), and 7% of polyvinyl chloride (PVC). Because of the absence of the efficient, clean and large-scale technology, the waste plastics have emerged as an environmental challenge that needs to be solved in the world.

Landfilling, incineration and recycling are the three main methods for treating the municipal plastic solid wastes. Because of the different national conditions, countries are different. The United States of America (USA) mainly utilizes the landfilling method, while the Europe and Japan mainly adopt the incineration method. At present, the rapid pyrolysis/catalytic pyrolysis liquefaction or chemical production of waste plastics has attracted the focus of attention in China and foreign countries, and is also the waste plastic treatment technology with the most promising industrialization prospects.

The rapid pyrolysis/catalytic pyrolysis of waste plastics is an effective recycling means involving with heating and decomposing waste plastics at high temperatures under oxygen-free or low-oxygen conditions and producing valuable products, such as the pyrolysis and liquefaction of waste plastics to produce gasoline, diesel oil, petroleum wax and other chemical raw materials. However, in view of the characteristics of waste plastics such as large size and light weight, difficulty in compression, poor air permeability, easy expansion, prone to melting and bonding, and poor thermal conductivity, it is difficult to solve the problem of continuous feeding and rapid heating. At present, most of the pyrolysis and liquefaction of waste plastics adopt reaction kettle-type intermittent reactions and horizontal type rotary kiln reactions, with the defects of slow heating rate, severe feedstock adhesion and coking, low liquid product yield and added value, difficult to improve processing capacity, difficult to scale up the pyrolysis device, and the subsequent processing of chlorine-containing liquid products is challenging, the defects have hampered the efficient, clean and large-scale utilization of waste plastics. There is no large-scale waste plastic pyrolysis device in commercial operation at present, it is urgent to develop a process and a equipment technology for producing wax-based chemicals and α-olefins from waste plastics based on self-heating melt dechlorination.

### SUMMARY

The present disclosure aims to overcome the defects in the prior art with respect to the pyrolysis liquidation technology of waste plastics, and provides a process for producing wax-based chemicals and α-olefins from waste plastics based on self-heating melt dechlorination. By means of the waste plastic pyrolysis dry gas and the high-temperature oil slurry circulation to carry out hot-melt dechlorination desulfurization pretreatment and atomizing feedstock, the problems that it is difficult to feed the pyrolysis feedstock and it is difficult to remove chlorine and sulfur heteroatoms are solved; the catalytic pyrolysis regulates and controls the C-C bond breaking, suppresses polycondensation and coking, and achieves controllable cracking of waste plastics; an use the downer reactor suppresses the secondary cracking, eliminates the magnification effect, maximize the production of wax-based chemicals and α-olefins, increases the temperature rise rate of the catalytic pyrolysis, solves the five common problems in the industry, namely adhesion and coking of waste plastic feedstock, difficulty in mixing the waste plastics with a heat carrier, low yield and added value of liquid product, difficulty in removing chlorine and sulfur heteroatoms from liquid product thereby affecting the subsequent processing, and difficulty in amplifying the pyrolysis device, thereby achieving the efficient, clean, high value and large-scale utilization of waste plastics.

The first aspect of the present disclosure provides a process for producing wax-based chemicals and α-olefins from waste plastics based on self-heating melt dechlorination, wherein the sorted clean fragments of PE and PP waste plastics are added into a water-cooling feed pipe at a temperature of 20-95° C. through a controllable feeder, and carried by a pyrolysis dry gas to fall into an oil slurry molten liquid at a temperature of 240-320° C. in a hot melting dechlorination reactor, and subjected to a molten dechlorination desulfurization pretreatment by stirring and mixing; a part of molten plastic liquid extracted from the bottom of said hot melting dechlorination reactor is heated by a high speed circulating tube equipped with an external heating to a temperature 20-60° C. higher than that of the oil slurry molten liquid, and then returned to the upper part of said hot melting dechlorination reactor, the other part of the molten plastic liquid is used as a raw material and conveyed to the upper middle part of a downer bed catalytic pyrolysis reactor to be fed through an atomizing feedstock nozzle; a fuel gas extracted from the top of the hot melting dechlorination reactor is subjected to a desulfurization dechlorination treatment and used as the fuel gas of a riser regenerator, wherein the flow rate of said molten plastic liquid in the high speed circulating tube is within the range of 2-30 m/s, a mass ratio of the circulation amount of the molten plastic liquid to the raw material of the hot melting dechlorination reactor is (1-10):1, and the steam dosage of the atomizing feedstock nozzle is 4 wt %-12wt % of the raw material dosage of the molten plastic liquid; the atomized particle feedstock of the molten plastic liquid is subjected to rapid mixing, vaporization and controlled cracking reaction with a regenerated catalyst having a temperature within the range of 600-800° C. falling from the top of said downer bed catalytic pyrolysis reactor, wherein the reaction temperature is within the range of 410-550° C., the pressure is a slight negative pressure, the reaction time is from 200 milliseconds to 2 seconds, maximize the production of wax-based chemicals and α-olefins, and a gas-solid separation is carried out at the bottom of said downer bed catalytic pyrolysis reactor to obtain a regenerated catalyst, and the pyrolysis oil and gas with a temperature of 410-550° C.; the separated regenerated catalyst passes through a return device and flows into the bottom of a riser regenerator, and subjected to an air fluidized coke burning regeneration at a temperature range of 500-750° C. and a fuel gas combustion-supporting heat supply, the coarse particle regenerated catalyst separated by a first gas-solid separator at the top of said riser regenerator is circulated back to the top of said downer bed catalytic pyrolysis reactor to catalytically pyrolyze the waste plastics, a fine catalyst separated by a second stage gas-solid separator is recycled back into the bottom of said riser regenerator to continue the fluidized coke burning regeneration, the fine ash is discharged via an ash discharge outlet, the flue gas is subjected to recovery of the waste heat by a waste heat boiler and then discharged outwardly; the separated pyrolysis oil gas with a temperature of 410-550° C. flows into a fractionating tower and is separated into α-olefin fraction, light wax oil, heavy wax oil, high temperature oil slurry and pyrolysis dry gas, 3v %-20v % of the pyrolysis dry gas is circulated back to the water-cooling feed pipe to be used as a carrying gas, and the high temperature oil slurry at a temperature larger than or equal to 350° C. is directly recycled to the upper part of said hot melting dechlorination reactor to be used as a melting heat source.

Through the above technical scheme, the present disclosure produces the favorable effects as follows:

By means of the waste plastic pyrolysis dry gas and the high-temperature oil slurry circulation to carry out hot-melt dechlorination desulfurization pretreatment and atomizing feedstock, the problems that it is difficult to feed the pyrolysis feedstock and it is difficult to remove chlorine and sulfur heteroatoms are solved; the catalytic pyrolysis regulates and controls the C—C bond breaking, suppresses polycondensation and coking, and achieves controllable cracking of waste plastics; an use the downer reactor suppresses the secondary cracking, eliminates the magnification effect, maximize the production of wax-based chemicals and α-olefins, increases the temperature rise rate of the catalytic pyrolysis, solves the five common problems in the industry, namely adhesion and coking of waste plastic feedstock, difficulty in mixing the waste plastics with a heat carrier, low yield and added value of liquid product, difficulty in removing chlorine and sulfur heteroatoms from liquid product thereby affecting the subsequent processing, and difficulty in amplifying the pyrolysis device, thereby achieving the efficient, clean, high value and large-scale utilization of waste plastics.

### BRIEF DESCRIPTION OF THE DRAWINGS

FIG. 1 illustrates an engineering process schematic diagram of the process for producing wax-based chemicals and α-olefins from waste plastics based on self-heating melt dechlorination according to the present disclosure.

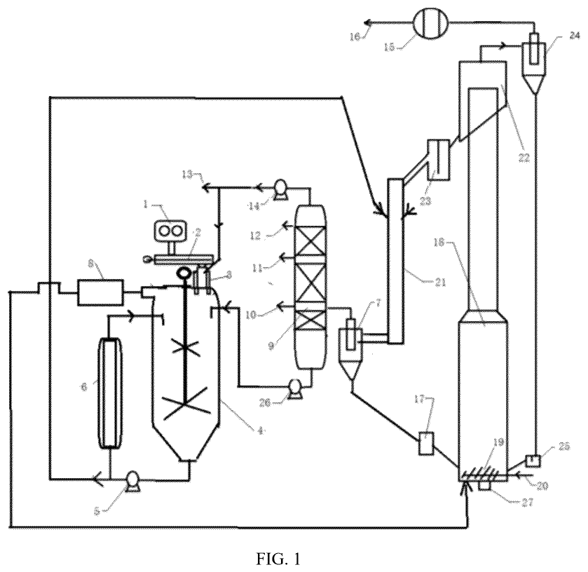

### DESCRIPTION OF REFERENCE SIGNS

1. Waste plastics pulverizer
2. Controllable feeder
3. Water-cooling feed pipe
4. Hot melting dechlorination reactor
5. Delivery pump
6. High speed circulating tube
7. Oil gas gas-solid separator
8. Desulfurization dechlorination reactor
9. Fractionating tower
10. Heavy wax oil outlet
11. Light wax oil outlet
12. α-olefin fraction outlet
13. Pyrolysis dry gas outlet
14. Induced draft fan
15. Waste heat boiler
16. Flue gas outlet
17. Regenerated catalyst return device
18. Riser regenerator
19. Gas distributor
20. Intake pipe
21. Downer bed catalytic pyrolysis reactor
22. First stage gas-solid separator
23. First stage coarse catalyst return feeder
24. Second stage gas-solid separator
25. Second stage fine catalyst return feeder
26. Delivery pump
27. Ash discharge outlet

The technological characteristics of the present disclosure will be described in detail below with reference to the attached drawings and examples.

### DETAILED DESCRIPTION

The terminals and any value of the ranges disclosed herein are not limited to the precise ranges or values, such ranges or values shall be comprehended as comprising the values adjacent to the ranges or values. As for numerical ranges, the endpoint values of the various ranges, the endpoint values and the individual point values of the various ranges, and the individual point values may be combined with one another to produce one or more new numerical ranges, which should be deemed to have been specifically disclosed herein.

A process for producing wax-based chemicals and α-olefins from waste plastics based on self-heating melt dechlorination, wherein the sorted clean PE and PP waste plastics are crushed by a waste plastics pulverizer 1, the clean fragments of PE and PP waste plastics are added into a water-cooling feed pipe 3 at a temperature of 20-95° C. through a controllable feeder 2, and carried by a pyrolysis dry gas to fall into an oil slurry molten liquid at a temperature of 240-320° C. in a hot melting dechlorination reactor 4 equipped with a stirrer, and subjected to a molten dechlorination desulfurization pretreatment by stirring and mixing, a molten plastic liquid extracted from the bottom of said hot melting dechlorination reactor 4 is pressurized and conveyed by delivery pump 5, a part of the molten plastic liquid is heated by a high speed circulating tube 6 equipped with an external heating to a temperature 20-60° C. higher than that of the oil slurry molten liquid, and then returned to the upper part of said hot melting dechlorination reactor 4, the other part of the molten plastic liquid is used as a raw material and conveyed to the upper middle part of a downer bed catalytic pyrolysis reactor 21 to be fed through an atomizing feedstock nozzle; a fuel gas extracted from the top of the hot melting dechlorination reactor 4 is subjected to the treatment by a desulfurization dechlorination reactor 8 and used as the fuel gas of a riser regenerator 18, wherein the flow rate of said molten plastic liquid in the high speed circulating tube is within the range of 2-30 m/s, a mass ratio of the circulation amount of the molten plastic liquid to the raw material of the hot melting dechlorination reactor is (1-10):1, and the steam dosage of the atomizing feedstock nozzle is 4 wt %-12 wt % of the raw material dosage of the molten plastic liquid; the atomized particle feedstock of the molten plastic liquid is subjected to the rapid mixing, vaporization and controllable cracking reaction with a regenerated catalyst having a temperature within the range of 600-800° C. falling from the top of said downer bed catalytic pyrolysis reactor 21, wherein the reaction temperature is within the range of 410-550° C., the pressure is a slight negative pressure, the reaction time is from 200 milliseconds to 2 seconds, maximize the production of wax-based chemicals and α-olefins, and a gas-solid separation is carried out by an oil gas gas-solid separator 7 at the bottom of said downer bed catalytic pyrolysis reactor 21 to obtain a regenerated catalyst, and the pyrolysis oil and gas with a temperature of 410-550° C.; the separated regenerated catalyst passes through a regenerated catalyst return device 17 and flows into the bottom of a riser regenerator 18, and subjected to an air fluidized coke burning regeneration introduced via a gas distributor 19 connected with an intake pipe 20 at a temperature range of 500-750° C. and a fuel gas combustion-supporting heat supply, the coarse particle regenerated catalyst separated by a first stage gas-solid separator 22 at the top of said riser regenerator 18 is circulated back via a first stage coarse catalyst return feeder 23 to the top of said downer bed catalytic pyrolysis reactor 21 to catalytically pyrolyze the waste plastics, a fine catalyst separated by a second stage gas-solid separator 24 is recycled back via a second stage fine catalyst return feeder 25 to the bottom of said riser regenerator 18 to continue the fluidized coke burning regeneration, the fine ash is discharged via an ash discharge outlet 27, the flue gas drained via a flue gas outlet 16 is subjected to recovery of the waste heat by a waste heat boiler 15 and then discharged outwardly; the pyrolysis oil gas with a temperature of 410-550° C. flows into a fractionating tower 9 and passes through a heavy wax oil outlet 10, a light wax oil outlet 11, an α-olefin fraction outlet 12, and a pyrolysis dry gas outlet 13 and is separated into α-olefin fraction, light wax oil, heavy wax oil, pyrolysis dry gas and high temperature oil slurry, 3v %-20v % of the pyrolysis dry gas is circulated back under the action of an induced draft fan 14 to the water-cooling feed pipe 3 to be used as a carrying gas, the remaining pyrolysis dry gas is discharged, and the high temperature oil slurry at a temperature larger than or equal to 350° C. is directly recycled via a delivery pump 26 to the upper part of said hot melting dechlorination reactor 4 to be used as a melting heat source.

In the present disclosure, the oil slurry molten liquid with a temperature of 240-320° C. is exactly the high temperature oil slurry circulated from the fractionating tower 9 to the upper portion of the hot melting dechlorination reactor 4.

According to the present disclosure, preferably, the outer wall of the water-cooling feed pipe 3 is provided with a water-cooling jacket; the water-cooling feed pipe 3 has a positive conical shape, a straight-pipe, or a reverse conical shape, more preferably a positive conical shape.

According to the present disclosure, preferably, the controllable feeder 2 is a rotary feeder, a dual-axis screw feeder or a single-axis screw feeder.

According to the present disclosure, preferably, the external heating of the high speed circulating tube 6 is performed by electromagnetic induction heating, heat conductive oil heating, steam heating or resistive heating, more preferably electromagnetic induction heating.

According to the present disclosure, the riser regenerator 18 is composed of a turbulent fluidized bed at the lower part and a carrying reactor at the upper part, the turbulent fluidized bed has a large equivalent diameter, the carrying reactor has a small equivalent diameter, the ratio of the equivalent diameter of said turbulent fluidized bed to the equivalent diameter of said carrying reactor is (2-3):1; the carrying reactor may be a straight tube reactor with uniform equivalent diameter, or is composed of large and small straight tubes with diameters of different multiples of the equivalent diameter connected by large and small head pipe fittings, and the diameter ratio of the large straight tube to the small straight tube is (1.2-2):1.

The process for producing wax-based chemicals and α-olefins from waste plastics based on self-heating melt dechlorination enables continuous, large-scale, efficient, clean and high value utilization of waste plastics, solves the five common problems in the industry, namely adhesion and coking of waste plastic feedstock, difficulty in mixing the waste plastics with a heat carrier, low yield and added value of liquid product, difficulty in amplifying the pyrolysis device, and difficulty in subsequent processing of the chlorine-containing liquid product, the yield of liquefied gas is larger than or equal to 10%, the yield of α-olefin fraction is larger than or equal to 20%, the yield of light wax oil fraction is larger than or equal to 40%, the yield of heavy wax oil fraction is larger than or equal to 25%, the overall yield of liquid products is 95% or more, which is increased by 25 or more percentage points over the conventional waste plastic pyrolysis technology, the advantages further comprise high pyrolysis strength, small equipment volume, low steel consumption, significantly reduced fixed investment, simple ash discharge process, thereby achieving the stable, reliable and long-term operation of the process plant.

The present disclosure will be described in detail below with reference to examples.

### Example 1

A process for producing wax-based chemicals and α-olefins from waste plastics based on self-heating melt dechlorination, wherein the fragments of PE and PP waste plastics were crushed by a waste plastics pulverizer 1 (dual-axis screw feeder), the clean fragments of PE and PP waste plastics were added into a water-cooling feed pipe 3 (has a positive conical shape, provided with a water-cooling jacket) at a temperature of 55° C. through a controllable feeder 2, and carried by a pyrolysis dry gas to fall into an oil slurry molten liquid at a temperature of 300° C. in a hot melting dechlorination reactor 4 equipped with a stirrer, and subjected to a molten dechlorination desulfurization pretreatment by stirring and mixing, a molten plastic liquid extracted from the bottom of said hot melting dechlorination reactor 4 was pressurized and conveyed by delivery pump 5, a part of the molten plastic liquid was heated by a high speed circulating tube 6 equipped with an electromagnetic induction external heating to a temperature 30° C. higher than that of the oil slurry molten liquid, and then returned to the upper part of said hot melting dechlorination reactor 4, the other part of the molten plastic liquid was used as a raw material and conveyed to the upper middle part of a downer bed catalytic pyrolysis reactor 21 to be fed through an atomizing feedstock nozzle; a fuel gas extracted from the top of the hot melting dechlorination reactor 4 was treated by a desulfurization dechlorination reactor 8 and used as the fuel gas of a riser regenerator 18, wherein the flow rate of said molten plastic liquid in the high speed circulating tube was 10 m/s, the ratio of the circulation amount of the molten plastic liquid to the raw material of the hot melting dechlorination reactor 4 was 4:1, and the steam dosage of the atomizing feedstock nozzle was 6 wt % of the raw material dosage of the molten plastic liquid; the atomized particle feedstock of the molten plastic liquid was subjected to the rapid mixing, vaporization and controllable cracking reaction with a regenerated catalyst having a temperature of 700° C. falling from the top of said downer bed catalytic pyrolysis reactor 21, wherein the reaction temperature was 500° C., the pressure was a slight negative pressure, the reaction time was 800 milliseconds, maximize the production of wax-based chemicals and α-olefins, and a gas-solid separation was carried out by an oil gas gas-solid separator 7 at the bottom of said downer bed catalytic pyrolysis reactor 21 to obtain a regenerated catalyst, and the pyrolysis oil and gas with a temperature of 500° C.; the separated regenerated catalyst passed through a regenerated catalyst return device 17 and flowed into the bottom of a riser regenerator 18, and subjected to an air fluidized coke burning regeneration introduced via a gas distributor 19 connected with an intake pipe 20 at a temperature of 700° C. and a fuel gas combustion-supporting heat supply; the coarse particle regenerated catalyst separated by a first stage gas-solid separator 22 at the top of said riser regenerator 18 was circulated back via a first stage coarse catalyst return feeder 23 to the top of said downer bed catalytic pyrolysis reactor 21 to catalytically pyrolyze the waste plastics, a fine catalyst separated by a second stage gas-solid separator 24 was recycled back via a second stage fine catalyst return feeder 25 to the bottom of said riser regenerator 18 to continue the fluidized coke burning regeneration, the fine ash was discharged via an ash discharge outlet 27, the flue gas drained via a flue gas outlet 16 was subjected to recovery of the waste heat by a waste heat boiler 15 and then discharged outwardly; the pyrolysis oil gas with a temperature of 500° C. flowed into a fractionating tower 9 and passed through a heavy wax oil outlet 10, a light wax oil outlet 11, an α-olefin fraction outlet 12, and a pyrolysis dry gas outlet 13 and was separated into α-olefin fraction, light wax oil, heavy wax oil, pyrolysis dry gas and high temperature oil slurry, the yield of liquefied gas was 11%, the yield of α-olefin fraction was 20%, the yield of light wax oil fraction was 40%, the yield of heavy wax oil fraction was 25%, the overall yield of liquid products was 96%; 15% of the pyrolysis dry gas was circulated back under the action of an induced draft fan 14 to the water-cooling feed pipe 3 to be used as a carrying gas, the remaining pyrolysis dry gas was discharged, and the high temperature oil slurry at a temperature of 350° C. was directly recycled via a delivery pump 26 to the upper part of said hot melting dechlorination reactor 4 to be used as a melting heat source.

The riser regenerator is composed of a turbulent fluidized bed at the lower part and a carrying reactor at the upper part, the ratio of the equivalent diameter of said turbulent fluidized bed to the equivalent diameter of said carrying reactor is 2.5:1, the carrying reactor is composed of large and small straight tubes connected by large and small head pipe fittings, and the diameter ratio of the large straight tube to the small straight tube is 1.5:1.

1. A process for producing wax-based chemicals and α-olefins from waste plastics based on self-heating melt dechlorination, wherein the sorted clean fragments of PE and PP waste plastics are added into a water-cooling feed pipe at a temperature of 20-95° C. through a controllable feeder, and carried by a pyrolysis dry gas to fall into an oil slurry molten liquid at a temperature of 240-320° C. in a hot melting dechlorination reactor, and subjected to a molten dechlorination desulfurization pretreatment by stirring and mixing; a part of molten plastic liquid extracted from the bottom of said hot melting dechlorination reactor is heated by a high speed circulating tube equipped with an external heating to a temperature 20-60° C. higher than that of the oil slurry molten liquid, and then returned to the upper part of said hot melting dechlorination reactor, the other part of the molten plastic liquid is used as a raw material and conveyed to the upper middle part of a downer bed catalytic pyrolysis reactor to be fed through an atomizing feedstock nozzle; a fuel gas extracted from the top of the hot melting dechlorination reactor is subjected to a desulfurization dechlorination treatment and used as the fuel gas of a riser regenerator, wherein the flow rate of the high speed circulating tube is within the range of 2-30 m/s, a mass ratio of circulation amount of the molten plastic liquid to the raw material of the hot melting dechlorination reactor is (1-10):1, and the steam dosage of the atomizing feedstock nozzle is 4wt %-12 wt % of the raw material dosage of the molten plastic liquid; the atomized particle feedstock of the molten plastic liquid is subjected to mixing, vaporization and cracking reaction with a regenerated catalyst having a temperature within the range of 600-800° C. falling from the top of said downer bed catalytic pyrolysis reactor, wherein the reaction temperature is within the range of 410-550° C., the pressure is within the range of 80-100 KPa (absolute pressure), the reaction time is from 200 milliseconds to 2 seconds, and a gas-solid separation is carried out at the bottom of said downer bed catalytic pyrolysis reactor; the separated regenerated catalyst passes through a return device and flows into the bottom of a riser regenerator and subjected to an air fluidized coke burning regeneration at a temperature range of 500-750° C. and a fuel gas combustion-supporting heat supply, a coarse particle regenerated catalyst separated by a first gas-solid separator at the top of said riser regenerator is circulated back to the top of said downer bed catalytic pyrolysis reactor to catalytically pyrolyze the waste plastics, a fine catalyst separated by a second stage gas-solid separator is recycled back into the bottom of said riser regenerator to continue the fluidized coke burning regeneration, flue gas is subjected to recovery of the waste heat by a waste heat boiler and then discharged outwardly; the separated pyrolysis oil gas with a temperature of 410-550° C. flows into a fractionating tower and is separated into α-olefin fraction, light wax oil, heavy wax oil, high temperature oil slurry and pyrolysis dry gas, 3 v %-20 v % of the pyrolysis dry gas is circulated back to the water-cooling feed pipe to be used as a carrying gas, and the high temperature oil slurry at a temperature larger than or equal to 350° C. is directly recycled to the upper part of said hot melting dechlorination reactor to be used as a melting heat source.

2. The process for producing wax-based chemicals and α-olefins from waste plastics based on self-heating melt dechlorination according to claim 1, wherein the outer wall of the water-cooling feed pipe is provided with a water-cooling jacket; the water-cooling feeding pipe has a positive conical shape, a straight-pipe, or a reverse conical shape.

3. The process for producing wax-based chemicals and α-olefins from waste plastics based on self-heating melt dechlorination according to claim 2, wherein the water-cooling feeding pipe has a positive conical shape.

4. The process for producing wax-based chemicals and α-olefins from waste plastics based on self-heating melt dechlorination according to claim 1, wherein the controllable feeder is a rotary feeder, a dual-axis screw feeder or a single-axis screw feeder.

5. The process for producing wax-based chemicals and α-olefins from waste plastics based on self-heating melt dechlorination according to claim 1, wherein the external heating of the high speed circulating tube is performed by electromagnetic induction heating, heat conductive oil heating, steam heating or resistive heating.

6. The process for producing wax-based chemicals and α-olefins from waste plastics based on self-heating melt dechlorination according to claim 5, wherein the external heating of the high speed circulating tube is performed by electromagnetic induction heating.

7. The process for producing wax-based chemicals and α-olefins from waste plastics based on self-heating melt dechlorination according to claim 1, wherein the riser regenerator is composed of a turbulent fluidized bed at the lower part and a carrying reactor at the upper part, the ratio of the equivalent diameter of said turbulent fluidized bed to the equivalent diameter of said carrying reactor is (2-3):1, the carrying reactor is a straight tube reactor, or is composed of large and small straight tubes connected by large and small head pipe fittings, and the diameter ratio of the large straight tube to the small straight tube is (1.2-2):1.
